(19) United States
(12) Patent Application Publication
(10) Pub. No.: US 2004/0176693 A1
(43) Pub. Date: Sep. 9, 2004

(54) Air pressure adjusting device

(76) Inventor: Chin-Jung Chen, Taipei (TW)

Correspondence Address: RABIN & Berdo, PC, 1101 14TH STREET, NW, SUITE 500, WASHINGTON, DC, 20005, US

(21) Appl. No.: 10/712,010

(22) Filed: Nov. 14, 2003

(30) Foreign Application Priority Data
Mar. 4, 2003 (TW) 92104598

(51) Int. Cl. A61B 5/02

(52) U.S. Cl. 600/495; 600/485; 600/490; 600/500

(57) ABSTRACT

An air pressure-adjusting device is provided for adjusting the air pressure in a container. A force is generated by the deflection of a deformation element in the device, which interacts with the force exerted on the deformation element by air in the container. As a result, the rate of lowering air pressure in the bladder of a blood pressure monitor may be controlled accurately.

This application claims the benefit of Taiwan application Serial No. 92104598, filed Mar. 4, 2003.

### BACKGROUND OF THE INVENTION

1. Field of the Invention

The invention relates in general to an air pressure adjusting device, and more particularly to an air pressure adjusting device, which uses a force on the outlet exerted as a result of the deformation of a deformation element to adjust the air pressure in a container.

2. Description of the Related Art

Studies have shown that, of all the hospital diagnostic tests, blood pressure tests are made the most frequently. However, blood-pressure measurements may be a little inaccurate. Typically, blood pressure is measured by using an aneroid monitor. By placing the stethoscope disk at the brachial artery, wrapping the bladder of the cuff snugly around the upper arm, and then squeezing the bulb to pump air into the cuff bladder, the mercury level of a blood pressure gauge will rise slowly. Pumping is stopped at a pressure of about 180 mmHg, temporarily stopping blood flow to the brachial artery. Air in the bladder is then released slowly at a stable rate (i.e. the bladder is deflated). Meanwhile, the mercury level in the pressure gauge is observed and heart sounds carefully monitored. When something that sounds like “prrpshh” (the sound of the heartbeat) is heard or appears for the first time, that is blood starts flowing in the artery again, the air pressure in the bladder is considered as the systolic blood pressure. The sounds continue and become louder in intensity. When the sound is heard for the last time or disappears, indicating a return to normal blood flow, the air pressure in the bladder is considered as the diastolic blood pressure. After the readings are taken, air in the bladder is released completely. Clearly, if air is released from the bladder too rapidly, blood pressure may not be determined. Therefore, an apparatus of controlling the release rate of air in the bladder will improve the precision of the readings from the pressure gauge.

Referring to FIG. 1A, a schematic view of a conventional air pressure-adjusting device with the shifting element adjoined to the bladder outlet is shown. The conventional air pressure-adjusting device 100 includes a driving element (not shown in FIG. 1A), a shifting element 110, and a deformation element 120.

The shifting element 110, coupled to the driving element (such as an electromagnetic coil), is driven to move along the shifting direction X1 by the driving element. The deformation element 120, disposed on the shifting element 110, will deform slightly as it touches the tip of the outlet 130 since the shifting element 110 moves to attach to the bladder outlet 130. Thus the tip of the outlet 130 sinks into the deformation element 120. At this time, the outlet 130 is covered by the deformation element 120, and thus air in the bladder may not be released through the outlet 130.

Referring to FIG. 1B, a schematic view of a conventional air pressure-adjusting device with the shifting element separated from the bladder outlet is shown. As described above, the outlet 130 is adjoined to the deformation element 120 so that air in the bladder may not be released from the outlet 130. If air in the bladder is to be released (i.e. the bladder is to be decompressed) so that the air pressure in the bladder may be lowered, the driving element should be used to drive the shifting element 110 to move away from the outlet 130 so that the deformation element 120 does not cover the outlet 130.

The shifting element 110, as driven by the driving element, may move back and forth along the shifting direction X1 as shown in FIG. 1A and FIG. 1B. The movement of the shifting element may force the deformation element 120 to cover the bladder outlet 130 and air in the bladder releases at a regular or desired rate. Therefore, air pressure in the bladder may be lowered steadily, or air in the bladder may be released completely.

As described above, in the conventional air pressure-adjusting device, the outlet 130 is either opened completely when not covered by the deformation element 120 or closed completely when covered by the deformation element 120. Therefore, in practice, the air pressure-adjusting device often has the following disadvantages. First, the rate of releasing air from the bladder is difficult to control accurately, because the outlet is either completely opened or closed. Secondly, air in the bladder may only be released in an erratic way. Therefore, the reduction of air pressure in the bladder may not be controlled effectively, and the air-releasing rate may not be regulated.

### SUMMARY OF THE INVENTION

It is therefore an object of the invention to provide an air pressure-adjusting device in order to accurately control the rate of air pressure reduction in the bladder of a blood pressure monitor.

The invention achieves the above-identified object by providing an air pressure-adjusting device in a container. The container includes an outlet for releasing air. The air pressure-adjusting device may be disclosed by three preferred embodiments as follows.

According to the first preferred embodiment, the air pressure-adjusting device includes a driving element, a shifting element, and a deformation element. The shifting element has a cavity and is coupled to the driving element. The shifting element may make a displacement when driven by the driving element. The deformation element, disposed at the cavity of the shifting element, is deflected due to the displacement of the shifting element. Accordingly, the deformation element may exert a force on the outlet. By adjusting this force and the air pressure in the bladder, the deformation element may control the amount of air released from the container and regulate the air pressure in the container.

According to the second preferred embodiment, the air pressure-adjusting device includes: a driving element, a shifting element, and a deformation element. The shifting element, coupled to the driving element, may make a displacement when driven by the driving element. The deformation element, having a chamber, is disposed in the outlet and coupled to the shifting element. There exists a gap between the deformation element and the inner wall of the outlet. Air in a container may be released out of the outlet via the gap. The chamber deforms according to the displacement of the shifting element, so that the deformation element may be deflected to close the gap. As a result of the changing equilibrium between the force generated by the deflection of the deformation element and the air pressure in the container, the deformation element may control the amount of air released via the gap and thus control the air pressure in the container. In practical application, the deformation element may have no chamber, which may also achieve the purpose of the invention.

According to the third preferred embodiment, the air pressure-adjusting device includes a driving element, a shifting element, and a deformation cover. The shifting element, coupled to the driving element, makes a displacement when driven by the driving element. The deformation cover, having a vent, is disposed to cover the outlet. Air in the container may be released from the outlet via the vent. Due to the displacement of the shifting element, the deformation cover is deformed and attaches to the outlet. Accordingly, the deformation cover may exert a force on the outlet. By adjusting the balance between this force and the air pressure in the container, the deformation cover may control the amount of air released out of the container and regulate air pressure in the container.

Other objects, features, and advantages of the invention will become apparent from the following detailed description of the preferred but non-limiting embodiments. The following description is made with reference to the accompanying drawings.

### BRIEF DESCRIPTION OF THE DRAWINGS

FIG. 1A (Prior Art) is a schematic view of a conventional air pressure adjusting device with the shifting element adjoining the bladder outlet;

FIG. 1B (Prior Art) is a schematic view of a conventional air pressure adjusting device with the shifting element separated from the bladder outlet;

FIG. 2A is a schematic view of the air pressure adjusting device according to the first preferred embodiment of the invention;

FIG. 2B is a schematic view of the movement of the shifting element along a shifting direction to reduce the deflection of the deformation element in a decompressing process;

FIG. 2C is a schematic view of the deformation of the deformation element to cover the outlet again with the shifting element located at that position as in FIG. 2B;

FIG. 3A is a schematic view of the air pressure adjusting device according to the second preferred embodiment of the invention;

FIG. 3B is a schematic view of the movement of the shifting element along a shifting direction to reduce the deflection of the deformation element in a decompressing process;

FIG. 3C is a schematic view of the deformation of the deformation element to close the gap again with the shifting element located at that position as in FIG. 3B;

FIG. 4A is a schematic view of the air pressure adjusting device according to the third preferred embodiment of the invention;

FIG. 4B is a schematic view of the movement of the shifting element along a shifting direction to reduce the deflection of the deformation cover in a decompressing process; and

FIG. 4C is a schematic view of the deformation of the deformation cover to cover the outlet again with the shifting element located at that position as in FIG. 4B.

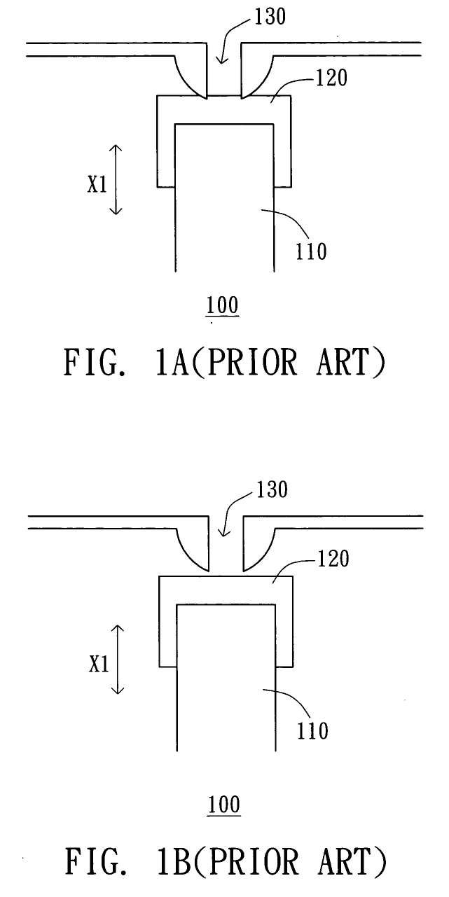

### DETAILED DESCRIPTION OF THE INVENTION

The deformation of the deformation element, according to the air pressure-adjusting device of the present invention, provides a force to interact with the air pressure in a container. Both the force exerted by the deformation element onto the outlet and the air pressure controls the release amount of the air and the consequential air pressure. The air pressure-adjusting device of the invention may be applied to any apparatus requiring the control of a gas or liquid releasing process, such as a blood pressure monitor. However, of course, this invention is not limited to be applied to a blood pressure monitor.

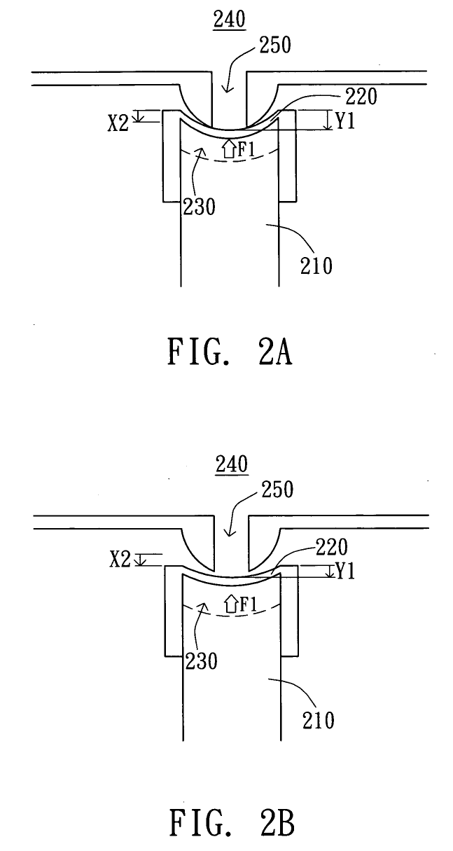

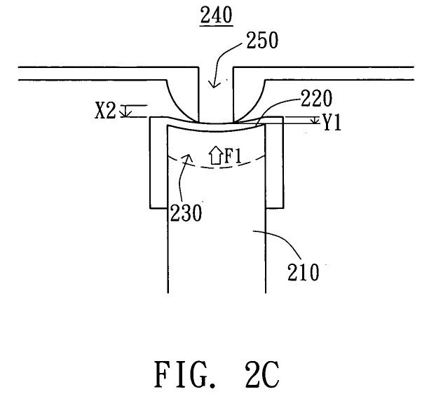

Referring to FIG. 2A, a schematic view of the air pressure-adjusting device according to the first preferred embodiment of the invention is shown. The air pressure-adjusting device includes a driving element (not shown in FIG. 2A), a shifting element 210 having a cavity 230, and a deformation element 220. The air pressure-adjusting device is applicable to the adjustment of air pressure in a container 240 having an outlet 250 for releasing air.

The shifting element 210, coupled to the driving element, may move along a shifting direction X2 by a displacement when driven by the driving element. The deformation element 220 is disposed on the cavity 230 of the shifting element 210.

At first, the deformation element 220 will be driven by the shifting element 210 to cover the outlet 250 and deform at the cavity 230. As shown in FIG. 2A, the deformation element 220 will exert a force F1 on the outlet 250 corresponding to its deflection Y1. As the deformation element 220 abuts more and more closely to the outlet 250, the deflection Y1 of the deformation element 220 will be increase. Therefore, the force F1 exerted on the outlet 250, which is generated by the deflection Y1, will also increase until it is larger than the force exerted on the deformation element 220 by air pressure in the container 240. At this time, the deformation element 220 may cover and seal the outlet 250 completely to prevent air releasing out of the container 240.

Referring to FIG. 2B, a schematic view of the movement of the shifting element along a shifting direction to reduce the deflection of the deformation element in a decompressing process is shown. Subsequently, as the air pressure in the container 240 is lowered, the shifting element 210 is driven by the driving element to make a displacement along the shifting direction X2 (away from the outlet 250), which reduces the deflection Y1 of the deformation element 220. Therefore, the force F1 exerted on the outlet 250 generated by the deflection Y1 of the deformation element 220 will be decreased. The deformation element 220 does not disengage from the outlet 250 until the force F1 is less than the force exerted on the deformation element 220 by air in the container 240. At this time, air in the container 240 may be released by way of the outlet 250 (as shown in FIG. 2B)

Referring to FIG. 2C, a schematic view of the deformation of the deformation element to again cover the outlet with the shifting element located at that position as in FIG. 2B is shown. In the decompressing process, air pressure in the container 240 is lowered gradually as air is released. The force exerted on the deformation element 220 decreases as the air pressure in the container 240 decreases and, at one point, it will be in equilibrium with the force F1 generated by the deflection Y1 of the deformation element 220. At this time, the deformation element 220 will again cover the outlet 250 and air in the container 240 will no longer be released.

As mentioned above, the driving element drives the shifting element 210 to move along the shifting direction X2 (away from the outlet 250) gradually. This increasing displacement reduces the deflection Y1 of the deformation element 220, thereby decreasing the force F1. On the other hand, decreasing air pressure in the container 240 reduces the force exerted on the deformation element 220 by air in the container 240. As a result of the changing balance between these two forces, the speed of releasing air out of the container 240 (i.e. the rate of reduction of air pressure in the container 240) may be regulated accurately. That is, in the decompressing process, the location of the shifting element 210 along the direction X2 may be quantified to represent the air pressure in the container 240 to some extent.

The container 240 in the invention may be a bladder of a blood pressure monitor, and the driving element may be any driving device, such as an electromagnetic coil, which may control and drive the movement of the shifting element.

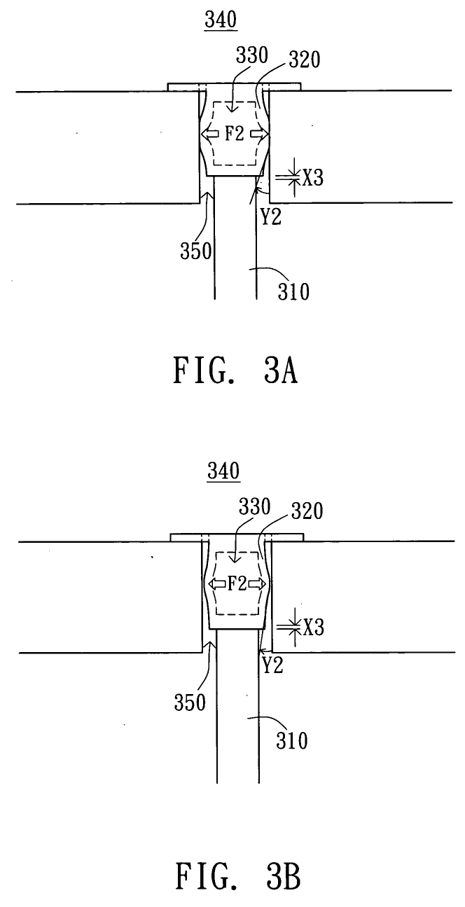

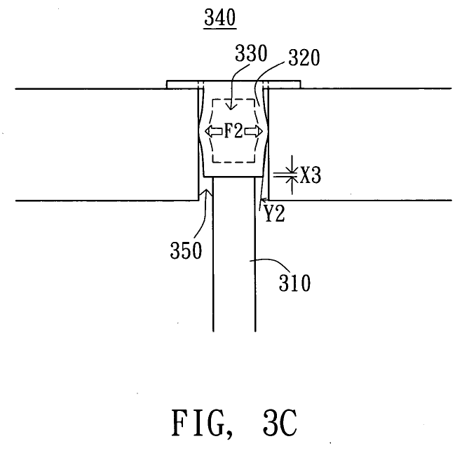

Referring to FIG. 3A, a schematic view of the air pressure-adjusting device according to the second preferred embodiment of the invention is shown. The air pressure-adjusting device includes a driving element (not shown in FIG. 3A), a shifting element 310, and a deformation element 320 having a chamber 330. The air pressure-adjusting device is used to regulate the air pressure in a container 340 with an outlet 350 for releasing air.

The shifting element 310, coupled to the driving element, may shift along a shifting direction X3 by a displacement, when driven by the driving element. The deformation element 320 is disposed in the outlet 350 and coupled to the shifting element 310. There exists a gap between the deformation element 320 and the inner wall of the outlet 350, and air in the container 340 may be released from the outlet 350 via the gap.

The chamber 330 may be forced to deflect due to the displacement of the shifting element 310 along the direction X3 (toward the deformation element 320). As shown in FIG. 3A, the chamber 330, when compressed by the shifting element 310, forces the deformation element 320 to deflect and close the gap. A force F2, generated by the deflection Y2 of the deformation element 320, is then exerted on the inner wall of the outlet 350. As the shifting element 310 moves closer to the container 340 by compressing the chamber 330, the chamber 330 will force the increased deflection Y2 of the deformation element 320. The force F2 exerted on the inner wall of the outlet 350 by the deformation element 320 then increases until it exceeds the force exerted on the deformation element 320 by air in the container 340. At this time, the deformation element 320 covers the outlet 350 to prevent the release of air out of the container 340.

Referring to FIG. 3B, a schematic view of the movement of the shifting element along a shifting direction to reduce the deflection Y2 of the deformation element in a decompressing process is shown. Subsequently, the shifting element 310 is driven by the driving element to make a displacement along the direction X3 (away from the outlet 350). This displacement results in the chamber 330 gradually recovers to its original shape, thereby reducing the deflection Y2 of the deformation element 320. Therefore, the force F2 exerted on the inner wall of the outlet 350 generated by the deflection Y2 of the deformation element 320 decreases until it is less than the force exerted on the sidewall of the deformation element 320 by air in the container 340. At this time, the deformation element 320 may not continue to be adjoined to the inner wall of the outlet 350, and air in the container 340 releases via the gap (as shown in FIG. 3B).

Referring to FIG. 3C, a schematic view of the deformation of the deformation element to again close the gap with the shifting element located at that position as in FIG. 3B is shown. In the decompressing process, air pressure in the container 340 gradually decreases as air is released. The force exerted on the deformation element 320 decreases because air pressure in the container decreases. Finally, air pressure is in equilibrium with the force F2 generated by the deflection Y2 of the deformation element 320. At this time, the deformation element 320 will again close the gap and air in the container 340 will no longer be released.

Thereafter, the driving element drives the shifting element 310 to move along the shifting direction X3 by a gradual displacement (away from the outlet 350). This increasing displacement reduces the stress on the chamber 330 and thus reduces the deflection Y2 of the deformation element 320, thereby decreasing the force F2. On the other hand, the decreasing of air pressure in the container 340 decreases the force exerted on the deformation element 320 by air in the container 340. As a result of the changing balance between these two forces, the speed of air release from the container 340 (i.e. the rate of lowering air pressure in the container 340) may be regulated accurately. That is, in the decompressing process, the location of the shifting element 310 along the direction X3 may be quantified to represent the air pressure in the container 340 to some extent.

In practical application, the deformation element 320 in the air pressure-adjusting device as mentioned above, may have no chamber 330. It is only necessary that a deflection Y2 may be generated at the deformation element 320 as it is squeezed by the shifting element 310. The container 340 as described above may be a bladder of a blood pressure monitor, and the driving element may be any driving device, such as an electromagnetic coil, which may control and drive the shifting element 310 to move.

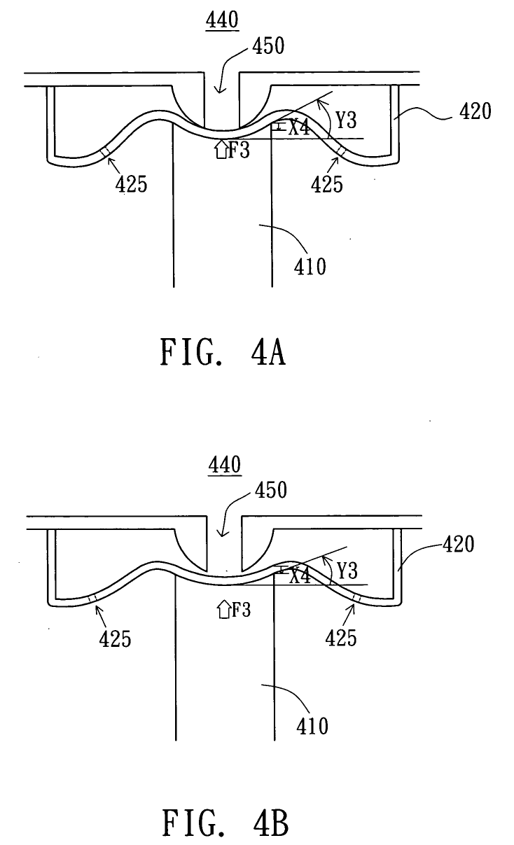

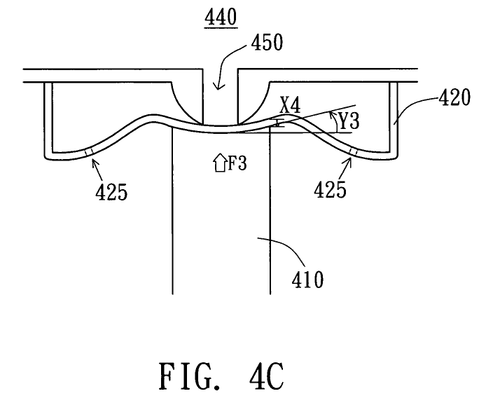

Referring to FIG. 4A, a schematic view of the air pressure-adjusting device according to the third preferred embodiment of the invention is shown. The air pressure-adjusting device includes a driving element (not shown in FIG. 4A), a shifting element 410, and a deformation cover 420 having a vent 425. The air pressure-adjusting device is used to regulate the air pressure in a container 440, which has an outlet 450 for releasing air.

The shifting element 410, coupled to the driving element, may shift along a shifting direction X4 by a displacement, as driven by the driving element. The deformation cover 420 is disposed to cover the outlet 450. Before the deformation cover 420 deflects, air in the container 440 may be released from the outlet 450 via the vent 425.

By the displacement of the shifting element 410, the deformation cover 420 may be deflected toward to the outlet 450. As shown in FIG. 4A, the deformation element 420 will exert a force F3 on the outlet 450 due to its deflection Y3. As the deformation cover 420 is forced by the shifting element 410 to adjoin more and more closely to the outlet 450, the deflection Y3 of the deformation cover 420 increases. Therefore, the force F3 exerted on the outlet 450 generated by the deflection Y3 increases until it is larger than the force exerted on the deformation cover 420 by air in the container 440. At this time, the deformation cover 420 may cover the outlet 450 to prevent air release from the container 440.

Referring to FIG. 4B, a schematic view of the movement of the shifting element along a shifting direction to reduce the deflection of the deformation cover in a decompressing process is shown. Subsequently, the shifting element 410 is driven by the driving element to make a displacement gradually along the direction X4 (away from the outlet 450). That is, the deflection Y3 of the deformation cover 420 is reduced. Thereafter, the force F3 exerted on the outlet 450 generated by the deformation Y3 of the deformation cover 420 decreases until it is less than the force exerted on the deformation cover 420 by air in the container 440. At this time, the deformation cover 420 may no longer adjoin the outlet 450, and air in the container 440 may be released via the vent 425 (as shown in FIG. 4B).

Referring to FIG. 4C, a schematic view of the deformation of the deformation cover 420 to again cover the outlet with the shifting element located at that position as in FIG. 4B is shown. In the decompressing process, air pressure in the container 440 is gradually reduced. The force exerted on the deformation cover 420 decreases as the air pressure in the container 440 is lowered and, at one point, it will be in equilibrium with the force F3 generated by the deflection Y3 of the deformation cover 420. At this time, the deformation cover 420 will again cover the outlet 450 and air in the container 440 will no longer be released.

The driving element drives the shifting element 410 to gradually move away from the outlet 450 along the shifting direction X4. This increasing displacement reduces the deflection Y3 of the deformation cover 420, thereby decreasing the force F3. On the other hand, the decreasing of air pressure in the container 440 reduces the force exerted on the deformation cover 420 by air in the container 440. As a result of the balance between these two forces, the speed of releasing air out of the container 440 (i.e. the rate of reduction of the air pressure in the container 440) may be regulated accurately, and the air pressure in the container may be controlled accurately. That is, in the decompressing process, the location of the shifting element 410 along the direction X4 may be quantified to represent the air pressure in the container 440 to some extent.

The container 440 as described above may be a bladder of a blood pressure monitor, and the driving element may be any driving device, such as an electromagnetic coil, which may control and drive the shifting element to move.

As described above, the main feature of the air pressure-adjusting device in the invention is that the deformation element may be forced to make a deflection when compressed by the shifting element, which generates a corresponding force on the outlet. As a result of the changing balance between the force exerted by the deformation element and the force exerted on the deformation element by air in the container, air pressure in the container may be regulated very accurately. Therefore, the lowering rate of air pressure in the container may be also controlled very accurately. In the typical decompressing process, the outlet may be opened and closed repeatedly by driving the shifting element to move back and forth, so air in the bladder may be released and air pressure in the cuff may be regulated. In this invention, as the container is to be decompressed, the shifting element is driven to move along the shifting direction away from the outlet only, not to move back and forth as in the prior art. The air pressure is adjusted by the different degrees of deflection of the deformation element resulting from the one-way displacement of the shifting element. By the deflection of the deformation element, air in the bladder may be controlled to release more steadily, and the air pressure in the bladder may be regulated more precisely. In other words, in the decompressing process, the displacement of the shifting element may be quantified to represent the air pressure in the bladder to some extent.

Noticeably, the geometrical structures of the elements in the air pressure-adjusting device are not limited to those in the preferred embodiments of the invention. One who is skilled in the art may use other geometrical structures as long as the purpose of adjusting air pressure accurately may be achieved, which will not depart from the spirit of the invention.

The air pressure-adjusting device according to the preferred embodiments of the invention has the following advantage: the lowering rate of air pressure in the bladder may be controlled accurately. Air in the container may be controlled to release constantly, that is, the process for controlling the rate of releasing air may be according to the air pressure-adjusting device in the invention.

While the invention has been described by way of example and in terms of a preferred embodiment, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. An air pressure-adjusting device, for adjusting an air pressure of a container, the container having an outlet for releasing air, the device comprising:
a driving element;
a shifting element, having a cavity, wherein the shifting element is coupled to the driving element, and is driven by the driving element to make a displacement; and
a deformation element, disposed at the cavity of the shifting element;
wherein the deformation element exerts a force to cover partially or completely the outlet or deform into the cavity according to a degree of the displacement of the shifting element, and the deformation element controls the amount of air released from the container to adjust an air pressure in the container according to the force and the air pressure in the container.

2. The air pressure-adjusting device according to claim 1, wherein the container is a bladder in a blood pressure monitor.

3. The air pressure-adjusting device according to claim 1, wherein the driving element is an electromagnetic coil.

4. An air pressure-adjusting device, for adjusting an air pressure in a container, the container having an outlet for releasing air, the device comprising:
a driving element;
a shifting element, coupled to the driving element, wherein the shifting element is driven to make a displacement by the driving element; and
a deformation element, having a chamber, the deformation element disposed in the outlet and coupled to the shifting element, wherein there exists a gap between the deformation element and an inner wall of the outlet, and air in the container is released from the outlet via the gap;
wherein the chamber deforms the deformation element to close partially or completely the gap according to a degree of the displacement of the shifting element, and the deformation element controls the amount of air released out of the gap to adjust an air pressure in the container according to the deformation and the air pressure in the container.

5. The air pressure-adjusting device according to claim 4, wherein the container is a bladder in a blood pressure monitor.

6. The air pressure-adjusting device according to claim 4, wherein the driving element is an electromagnetic coil.

7. An air pressure-adjusting device, for adjusting an air pressure of a container, the container having an outlet for releasing air, the device comprising:
a driving element;
a shifting element, coupled to the driving element, wherein the shifting element is driven to make a displacement by the driving element; and
a deformation element, disposed in the outlet and coupled to the shifting element, wherein there exists a gap between the deformation element and an inner wall of the outlet, and air in the container is released from the outlet via the gap;
wherein the deformation element is deflected to close partially or completely the gap according to a degree of the displacement of the shifting element, and the deformation element controls the amount of air released out of the gap to adjust an air pressure in the container according to the deformation and the air pressure in the container.

8. The air pressure-adjusting device according to claim 7, wherein the container is a bladder in a blood pressure monitor.

9. The air pressure-adjusting device according to claim 7, wherein the driving element is an electromagnetic coil.

10. An air pressure adjusting device, for adjusting an air pressure of a container, the container having an outlet for releasing air, the device comprising:
a driving element;
a shifting element, coupled to the driving element, wherein the shifting element is driven to make a displacement by the driving element; and
a deformation cover, having a vent, wherein the deformation cover is disposed on the outlet to cover the outlet, and air in the container is released from the outlet via the gap;
wherein the deformation cover is deflected and exerts a force to cover partially or completely the outlet according to a degree of the displacement of the shifting element, and the deformation cover controls the amount of air released from the container to adjust an air pressure in the container according to the force and the air pressure in the container.

11. The air pressure-adjusting device according to claim 10, wherein the container is a bladder in a blood pressure monitor.

12. The air pressure-adjusting device according to claim 10, wherein the driving element is an electromagnetic coil.
